(19) United States
(12) Patent Application Publication
(10) Pub. No.: US 2008/0120040 A1
(43) Pub. Date: May 22, 2008

(54) SYSTEM AND METHOD FOR PROVIDING CONTENTS USED IN ULTRASOUND DIAGNOSTIC DEVICE

(75) Inventors: Jae Gyoung Kim, Seoul (KR); Young Seuk Song, Seoul (KR); Mi Jeoung Ahn, Seoul (KR)

Correspondence Address: OBLON, SPIVAK, MCCLELLAND MAIER & NEUSTADT, P.C., 1940 DUKE STREET, ALEXANDRIA, VA, 22314, US

(73) Assignee: Medison Co., Ltd., Hongchun-gun (KR)

(21) Appl. No.: 11/857,888

(22) Filed: Sep. 19, 2007

(30) Foreign Application Priority Data
Nov. 20, 2006 (KR) 10-2006-0114653

(51) Int. Cl. G06F 19/00; G01N 33/48

(52) U.S. Cl. 702/19; 600/437

(57) ABSTRACT

The present invention is directed to a method of providing software contents through a communication network, comprising the steps of: (a) arranging a contents database to store a plurality of software contents in the contents providing system; (b) connecting the ultrasound diagnostic device to the contents providing system through the communication network; (c) uploading first software contents from the ultrasound diagnostic device to the contents providing system to store the first software contents in the contents database; and (d) upon a download request for second software contents from the ultrasound diagnostic device, retrieving the second software contents from the contents database to provide the second software contents to the ultrasound diagnostic device.

The present application claims priority from Korean Patent Application No. 10-2006-0114653 filed on Nov. 20, 2006, the entire subject matter of which is incorporated herein by reference.

### BACKGROUND

1. Field

The present invention generally relates to a system and method for providing contents to be used in an ultrasound diagnostic device.

2. Background

An ultrasound diagnostic device has a wide range of applications and has become one of the important diagnostic devices. Specifically, due to its non-invasive and non-destructive nature, the ultrasound diagnostic device has been extensively used in the medical profession. Modern high-performance ultrasound diagnostic devices are commonly used to produce two or three-dimensional images of internal features of an object.

Generally, an ultrasound diagnostic device comprises software for driving a tool and producing two or three-dimensional images of internal features of an object. Further, the ultrasound diagnostic device may comprise various tools for obtaining two or three-dimensional images of internal features of an object or for improving image quality of said images by modifying their image settings. These tools and software are provided by the manufacturer of the ultrasound diagnostic device or by a third party. Such tools and software can be updated with new versions.

When a new version of software is developed, the manufacturer of the ultrasound diagnostic device (or a third party) must typically visit the location where the ultrasound diagnostic device is installed (e.g., medical center) in order to install the new version to update the software included in the device. This requires a lot of time and effort. For example, although a new version of a tool is developed to reflect various guidelines for obtaining heart images presented by relevant societies such as ASE and ACC, it is difficult to share and manage the new version.

### BRIEF DESCRIPTION OF THE DRAWINGS

Arrangements and embodiments may be described in detail with reference to the following drawings in which like reference numerals refer to like elements and wherein:

FIG. 1 is a block diagram illustrating a system for providing contents to be used in an ultrasound diagnostic device in accordance with one embodiment of the present invention;

FIG. 2 is a flow chart showing procedures for providing contents in accordance with one embodiment of the present invention;

FIG. 3 is a flow chart showing procedures for downloading contents in accordance with one embodiment of the present invention; and

FIG. 4 is a flow chart showing procedures for uploading contents in accordance with one embodiment of the present invention.

### DETAILED DESCRIPTION OF THE PRESENT INVENTION

A detailed description may be provided with reference to the accompanying drawings. One of ordinary skill in the art may realize that the following description is illustrative only and is not in any way limiting. Other embodiments of the present invention may readily suggest themselves to such skilled persons having the benefit of this disclosure.

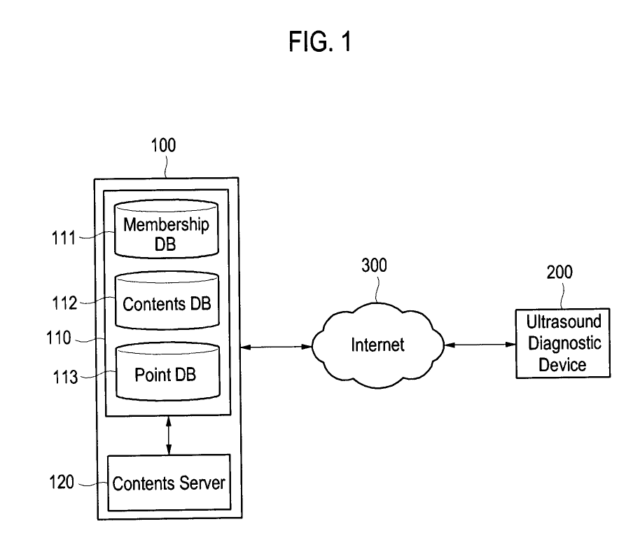

FIG. 1 illustrates a scheme for a system 100 adapted to provide contents to be used in an ultrasound diagnostic device in accordance with one embodiment of the present invention. As shown in FIG. 1, such a system 100 is connected to an ultrasound diagnostic device 200 through the Internet 300 and comprises a database 110 and a contents server 120. The Internet 300 should be interpreted and understood as a communication network wherein conventional wired and wireless communication networks are interconnected. The ultrasound diagnostic device 200 may be any device, which can obtain ultrasound images of an object.

As shown in FIG. 1, the database 110 comprises a membership database 111, a contents database 112 and a point database 113.

The membership database 111 stores information associated with members registered to the contents providing system 100 (“membership information”). In particular, the membership database 111 may store membership information including the names, addresses, phone numbers, e-mail addresses, IDs and passwords of the members using the ultrasound diagnostic device 200 and registered to the contents providing system 100.

The contents database 112 stores contents, which may be used in the ultrasound diagnostic device 200. For example, such contents may include the following: a software update to update the software for driving and operating the ultrasound diagnostic 200, as well as to obtain two or three-dimensional images of internal features of an object; a tool for setting a probe according to patient status and diagnosed parts; an image setting tool for obtaining optimal images by adjusting images according to the patient status and diagnosed parts; a measure table tool for each application; and a measure report. A measure table may include reference values for respective body parts of a fetus, which are classified according to weekly development stage.

The point database 113 stores information regarding the ID and points of each of the members.

The contents server 120 manages contents inputted to or outputted from the database 110. According to one embodiment of the present invention, the contents server 120 provides a web page for uploading and downloading contents to the ultrasound diagnostic device 200 connected to the contents providing system 100. The web page may be implemented with HTML (Hyper-Text Markup Language) and/or WML (Wireless Markup Language), which may be accessed by the ultrasound diagnostic device 200. The contents server 120 stores the contents uploaded from the ultrasound diagnostic device 200 in the contents database 112 and updates point data in the point database 113 by adding predetermined points to the points of the member who has uploaded the contents. The contents server 120 retrieves the contents, which the ultrasound diagnostic device 200 requests to download from the contents database 112, and obtains the contents to be downloaded to the ultrasound diagnostic device 200. Further, the contents server 120 updates point data in the point database 113 by subtracting predetermined points from the points of the member who has downloaded the contents. The contents server 120 further comprises a communication module (not shown) connected to the contents providing system 100 through the Internet 300.

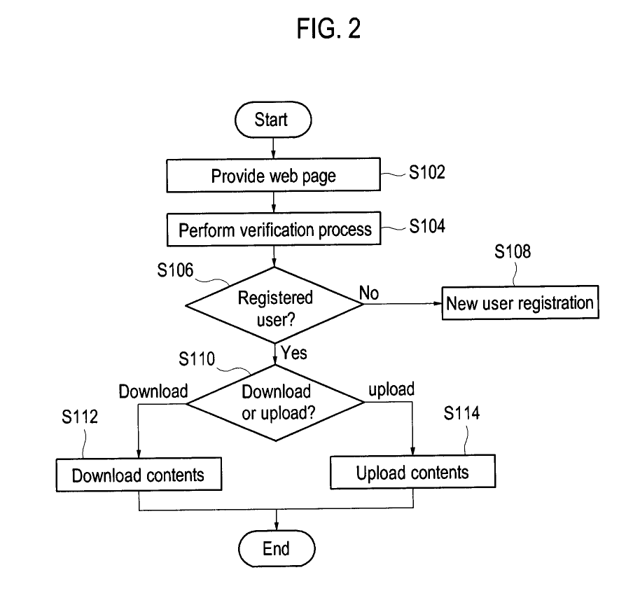

The procedures for providing contents in accordance with the embodiment of the present invention are explained below with reference to FIGS. 2-4.

As shown in FIG. 2, when the ultrasound diagnostic device connects to the contents providing system 100 through the Internet 300, the contents server 120 provides a web page for contents upload/download to the ultrasound diagnostic device 200 (S102) and then performs verification process on the ultrasound diagnostic device 200 (S104). The verification process is performed by comparing the ID and password entered into the ultrasound diagnostic device 200 with the membership information stored in the membership database 111.

The contents server 120 performs the verification process to determine whether the user of the ultrasound diagnostic device 200 is registered to the contents providing system 100 (S106). If the user of the ultrasound diagnostic device 200 is not a registered user, then the contents server 120 performs a new user registration process (S108). The new user registration process may be implemented by using well-known techniques. Thus, detailed explanations thereon are omitted herein.

If the user of the ultrasound diagnostic device 200 is a registered user, then the contents server 120 determines whether a predetermined menu arranged in the web page is selected (S110). For the sake of convenience, it is assumed that an upload menu for uploading contents to the contents providing system 100 and a download menu for downloading contents from the contents providing system 100 are included in the web page. However, one of ordinary skill in the art understands that various types of menus may be added to the web page as necessary.

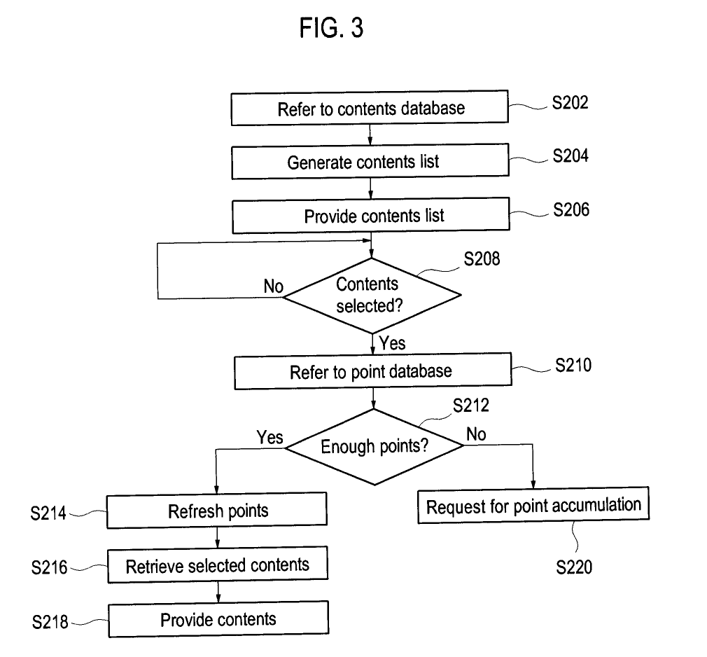

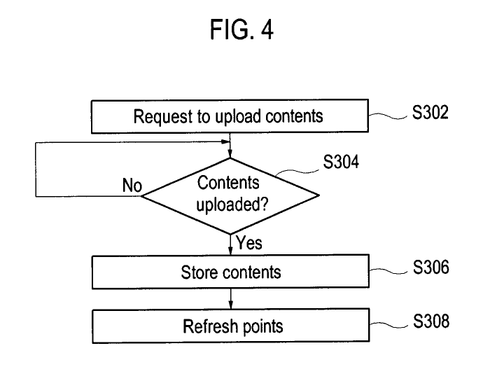

If it is determined that the download menu is selected in S110, then the contents server 120 performs a contents download process (S112). If it is determined that the upload menu is selected in S110, then the contents server 120 performs a contents upload process (S114). The steps S112 and S114 will be explained with reference to FIGS. 3 and 4.

As shown in FIG. 3, the contents server 120 refers to the contents database 112 (S202) and generates a list of contents stored in the contents database 112 (S204). Further, the contents server 120 provides the list to the ultrasound diagnostic device 200 (S206).

When some contents are selected by the ultrasound diagnostic device 200 (S208), the contents server 120 refers to the point database 113 (S210) and determines whether the user of the ultrasound diagnostic device 200 (i.e., registered member) has enough points for downloading the contents (S212).

If it is determined that the member does not have enough points for the download in S212, then the contents server 120 requests for point accumulation to the ultrasound diagnostic device 200 (S220). Such point accumulation may be performed by uploading contents or purchasing extra points for cash or credit cards. Since the point purchase for cash or credit cards may be implemented by using well-known purchase processes, detailed explanations thereon are omitted herein.

If it is determined that the member has enough points for downloading the contents in S212, then the contents server 120 reduces the points of the member who has requested to download the contents (S214). That is, the contents server 120 updates the member's points by subtracting predetermined contents download points from the points stored in the point database 113.

The contents server 120 retrieves the contents selected by the ultrasound diagnostic device 200 from the contents database 112 (S216) and provides the contents to the ultrasound diagnostic device 200. That is, the contents server obtains the contents to be downloaded to the ultrasound diagnostic device 200 through the Internet 300 (S218).

As shown in FIG. 4, the contents server 120 requests the ultrasound diagnostic device 200 to upload contents (S302). When contents are uploaded from the ultrasound diagnostic device 200 (S304), the uploaded contents are stored in the contents database 112 (S306) and predetermined points are given to the member who has uploaded the contents (S308). That is, the contents server 120 updates the member's points by adding predetermined contents upload points to the points stored in the point database 113.

In accordance with one embodiment of the present invention, there is provided a contents providing system accessible by an ultrasound diagnostic device through a communication network, which comprises: a database adapted to store a plurality of software contents; and a server operable to store first software contents uploaded from the ultrasound diagnostic device in said database, said server being responsive to a download request for second software contents from the ultrasound diagnostic device to retrieve the second software contents from said database for provision to the ultrasound diagnostic device.

In accordance with one embodiment of the present invention, there is provided a method of providing contents by using a contents providing system interconnected with an ultrasound diagnostic device through a communication network, which comprises: (a) arranging a contents database to store a plurality of software contents in the contents providing system; (b) connecting the ultrasound diagnostic device to the contents providing system through the communication network; (c) uploading first software contents from the ultrasound diagnostic device to the contents providing system to store the first software contents in the contents database; and (d) upon a download request for second software contents from the ultrasound diagnostic device, retrieving the second software contents from the contents database to provide the second software contents to the ultrasound diagnostic device.

Any reference in this specification to “one embodiment,” “an embodiment,” “example embodiment,” etc., means that a particular feature, structure or characteristic described in connection with the embodiment is included in at least one embodiment of the present invention. The appearances of such phrases in various places in the specification are not necessarily all referring to the same embodiment. Further, when a particular feature, structure or characteristic is described in connection with any embodiment, it is submitted that it is within the purview of one skilled in the art to effect such feature, structure or characteristic in connection with other ones of the embodiments.

Although embodiments have been described with reference to a number of illustrative embodiments thereof, it should be understood that numerous other modifications and embodiments can be devised by those skilled in the art that will fall within the spirit and scope of the principles of this disclosure. More particularly, numerous variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. A contents providing system accessible by an ultrasound diagnostic device through a communication network, comprising:
a database for storing a plurality of software contents; and
a server for storing first software contents uploaded from the ultrasound diagnostic device in said database, said server being responsive to a download request for second software contents from the ultrasound diagnostic device to retrieve the second software contents from said database and provide the same to the ultrasound diagnostic device.

2. The contents providing system of claim 1, wherein the plurality of software contents comprise software for driving and operating the ultrasound diagnostic device.

3. The contents providing system of claim 1, wherein said database comprises:
a membership database for storing information on members registered to said contents providing system;
a contents database for storing the first and second software contents; and
a point database for storing information on points of each of the members.

4. The contents providing system of claim 3, wherein said server generates a list of the software contents stored in said contents database so as to provide the same to the ultrasound diagnostic device; upon receiving contents selection information from the ultrasound diagnostic device, retrieves software contents corresponding to the contents selection information from said contents database so as to provide the same to the ultrasound diagnostic device; and subtracts predetermined download points for downloading the software contents from the points stored in said point database.

5. The contents providing system of claim 3, wherein said server stores the first software contents uploaded from the ultrasound diagnostic device in said contents database upon an upload request from the ultrasound diagnostic device and adds predetermined upload points for uploading the software contents to the points stored in said point database.

6. A method of providing contents by using a contents providing system interconnected with an ultrasound diagnostic device through a communication network, comprising:
(a) arranging a contents database to store a plurality of software contents in the contents providing system;
(b) connecting the ultrasound diagnostic device to the contents providing system through the communication network;
(c) uploading first software contents from the ultrasound diagnostic device to the contents providing system to store the first software contents in the contents database; and
(d) upon a download request for second software contents from the ultrasound diagnostic device, retrieving the second software contents from the contents database to provide the second software contents to the ultrasound diagnostic device.

7. The method of claim 6, wherein the plurality of software contents comprise software for driving and operating said ultrasound diagnostic device.

8. The method of claim 6, wherein the contents providing system comprises a point database for storing information on points of each of members registered to the contents providing system, and wherein the step (c) comprises:
(c1) requesting the ultrasound diagnostic device to upload the first software contents;
(c2) storing the first software contents uploaded from the ultrasound diagnostic device in the contents database;
(c3) adding predetermined upload points for uploading the first software contents to the points stored in the point database.

9. The method of claim 6, wherein the contents providing system comprises a point database for storing information on points of each of members registered to the contents providing system, and wherein the step (d) comprises:
(d1) generating a list of the software contents stored in the contents database;
(d2) providing the list to the ultrasound diagnostic device;
(d3) receiving contents selection information from the ultrasound diagnostic device;
(d4) retrieving software contents corresponding to the contents selection information from the contents database to provide the contents to the ultrasound diagnostic device; and
(d5) subtracting predetermined download points for downloading the contents from the points stored in the point database.
